(19) United States
(12) Patent Application Publication
(10) Pub. No.: US 2008/0039954 A1
(43) Pub. Date: Feb. 14, 2008

(54) Expandable cartilage implant

(75) Inventors: Marc Long, Denville, NJ (US); Twana Davisson, Montclair, NJ (US)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Howmedica Osteonics Corp., Mahwah, NJ (US)

(21) Appl. No.: 11/891,057

(22) Filed: Aug. 8, 2007

Related U.S. Application Data
(60) Provisional application No. 60/836,253, filed on Aug. 8, 2006.
(60) Provisional application No. 60/861,341, filed on Nov. 27, 2006.

(51) Int. Cl. A61F 2/02

(52) U.S. Cl. 623/23.76; 623/23.72

(57) ABSTRACT

The present invention relates to a method for repairing cartilage defects in a patient. According to the current invention, a porous material having at least expandable or compressible properties is implanted into a cartilage defect.

### CROSS REFERENCE TO RELATED APPLICATIONS

This application claims the benefit of U.S. Provisional Application No. 60/836,253, filed on Aug. 8, 2006 and U.S. Provisional Application No. 60/861,341 filed on Nov. 27, 2006, the disclosures of which are incorporated by reference herein.

### FIELD OF THE INVENTION

The present invention relates to the field of medical technology and is generally directed to the treatment of cartilage or cartilage and bone defects through the use of grafts.

### BACKGROUND OF THE INVENTION

Cartilage is an avascular connective tissue made up of collagen and/or elastin fibers, and chondrocytes, all of which are embedded in a matrix. There are three main types of cartilage: elastic, fibrocartilage, and hyaline. Elastic cartilage is found in the outer ear and the epiglottis. Fibrocartilage is found between the bones of the spinal column, hips and pelvis. Hyaline cartilage can be found on the ends of bones which form joints, on the ends of the ribs, on the end of the nose, on the stiff rings around the windpipe, and supporting the larynx. Articular cartilage is a specialized type of hyaline cartilage which covers the surface of joints and provides a durable low friction surface that distributes mechanical forces and protects the joint's underlying bone.

Different types of collagen can be found in varying amounts in the collagen matrix, depending on the type of tissue. For example, hyaline cartilage, which is found predominantly in articulating joints, is composed mostly of type II collagen with small amounts of types V, VI, IX, X, and XI collagen also present. On the other hand, fibrocartilage, which can also be found in joints, is primarily composed of type I collagen. Additionally, the fibrocartilaginous tissue that sometimes replaces damaged articular cartilage is composed of type I collagen.

Loss of or damage to cartilage can lead to painful conditions such as osteoarthritis. Damage to cartilage can be caused by traumatic injury, disease and/or age. Since cartilage lacks nerves and blood vessels, it has very limited regenerative capabilities compared to other tissues. Consequently, the healing of damaged joint cartilage results in a fibrocartilaginous repair tissue that lacks the structure and biomechanical properties of normal cartilage. Over time, the repair tissue degrades and leaves damaged joint cartilage, which causes osteoarthritis and reduced movement in the joint.

There is a need for methods for repairing cartilage defects.

### SUMMARY OF THE INVENTION

The present invention includes a graft that can be used to repair cartilage and methods of producing the graft. The invention also includes a method of treating cartilage defects using the graft.

The graft comprises a porous material that is also compressible and/or expandable. The graft can be used as both a scaffold for ex vivo cartilage growth or as an implant used to repair cartilage. The material used in the graft can be implanted alone or in combination with cells and/or biological factors at the time of surgery. The graft may be used for chondral, osteochondral, partial or full repair of cartilage defects.

### BRIEF DESCRIPTION OF THE DRAWINGS

FIG. 1 shows the compressive properties of the material of the invention.

FIG. 2 shows the expansion of the material of the invention in a cartilage defect.

FIG. 3 shows DBM graft filled defects (A and B) and autograft-filled defects (C) after 3 months.

FIG. 4 shows Safranin-O staining of cross sections of goat joints at 3 weeks post-implantation with DBM.

FIG. 5 shows the expansion of the material inside the defect.

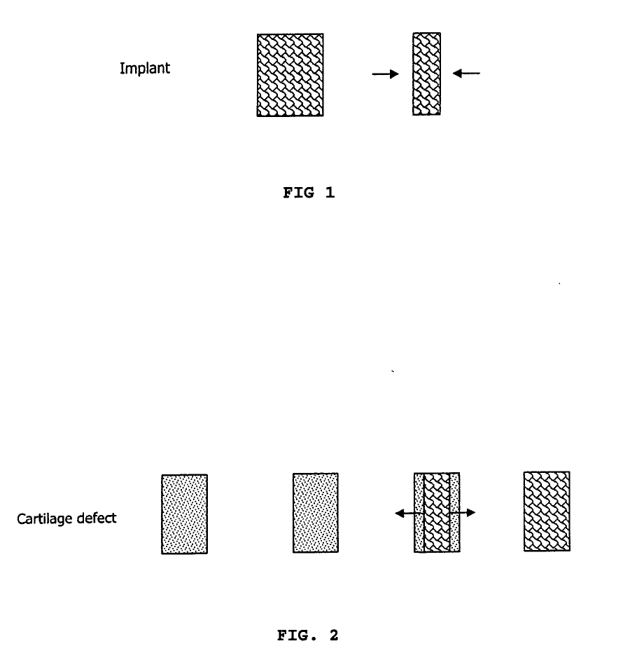

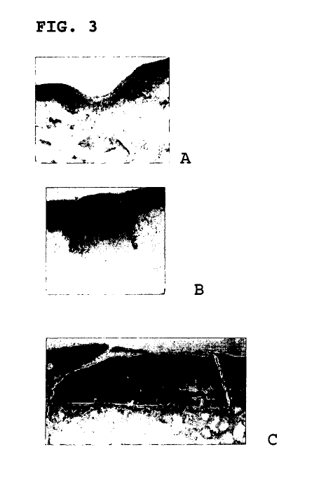

### DETAILED DESCRIPTION OF THE INVENTION

The present invention is directed to the repair of cartilage and includes a cartilage graft and a method of repairing a cartilage defect using the cartilage graft.

Although a number of different therapeutic methods are currently being used to treat cartilage defects, they have only been marginally successful. Some of the current treatments include lavage, arthroscopic debridement, and repair stimulation. However, these therapeutic methods either provide only temporary pain relief or have shown limited clinical efficacy.

Other treatment methods involve grafting the defect site with artificial materials, autografts, allografts, or xenografts. Examples of different grafts and grafting methods can be found in U.S. Patent and Application Nos. 5,944,755; 5,782,915; 6,858,042; 2003/0229400; and 2004/0230303, the disclosures of which are incorporated by reference herein. Grafts for cartilage repair include porous materials, such as PLA, collagen “sponges”, hyaluronic acid, metals (CoCr, Titanium), PVA, autograft, and allograft osteochondral plugs. None of these materials are both porous and expandable or compressible to a significant amount of their original size.

One particular grafting method, called mosaicplasty, has shown some clinical efficacy. Mosaicplasty involves removing small autologous osteochondral plugs from low weight bearing sites in a patient's joint. The osteochondral plugs are then grafted into a mosaic of holes drilled into the patient's articular cartilage defect site. Some patients who have undergone mosaicplasty have reported decreased pain and improved joint function. Marcacci, M. et al., Arthroscopy 21 (4): 462-470 (2005).

Although all of the above methods have had some clinical success, each one of these therapeutic methods suffer from one or more of the following disadvantages: the risk of patient immune response or disease transmission; limited availability of osteochondral autograft sites; lack of implant adhesion to the defect site; implant deterioration; lack of long-term efficacy; donor site morbidity; patient discomfort; and the failure to restore normal joint function.

The Osteosponge™ (Bacterin International, Inc.; Belgrade, Mont.) has been developed for bone defects. It is a porous, compressible and expandable demineralized bone matrix (DBM), which has been shown to be useful as a scaffold for bone repair. The present inventors have shown that the Osteosponge™ can also be used for cartilage repair. The compressible and expandable DBM sponge is porous and can be compressed to 30% of its size prior to implantation. See the following U.S. Publications and Issued Patents for similar products: 2006/0085075; 2005/0090899; 2004/0115240; 2004/0197375; 20040062753; 20040166169; 20040078090; U.S. Pat. Nos. 7,056,337; 6,121,042; 6,319,712; 6,171,610; 5,882,929; and 6,124,273.

In the present invention, a graft comprising a material with sponge-like properties similar to Osteosponge™ is used in the repair of cartilage defects (chondral or osteochondral). The graft allows cartilage growth, resulting in restoration of function.

Thus, the present invention is directed to a method for cartilage repair comprising implanting a graft into a cartilage defect site in a patient, wherein the graft comprises a porous material which is also expandable and/or compressible. The material is porous, to allow in-growth of cells. The material is also compressible and/or expandable for better press-fit and chondro-integration.

Integration with the surrounding cartilage tissue is not commonly achieved when a normal “press-fit” plug is used. A tighter press-fit should be achieved by the expansion of the material of the invention inside the defect, and will enhance integration and improve the performance of the cartilage implant.

The graft of the invention achieves better apposition with the surrounding cartilage tissue and decreases, or eliminates, micromotion. These results would be expected to yield improved healing of the cartilage defect and increased longevity. In addition, the graft will provide a scaffold with improved fixation due to its ability to be compressed and expand inside the defect.

The material should be porous enough to allow cell growth. Each pore may be the same size, or the pores may be of varying sizes, so long as some of the pores are large enough to allow cell growth into the material. Additionally, the pores may vary or change in size on compression and/or expansion of the material. In certain embodiments, the material has pores with a diameter of at least about 10 microns, at least about 20 microns, at least about 30 microns, at least about 40 microns or at least about 50 microns. Larger size pores are also within the scope of the invention, for example at least about 75-1000 microns.

The material used in the invention is expandable and/or compressible by a significant amount. By “expandable by a significant amount” it is meant that the materials expand by at least about 5 or 10% of their original size. By “compressible by a significant amount” it is meant that the materials compress by at least about 5 or 10% of their original size.

The material may expand by at least about 5 or 10% to at least about 300% of its original size. For example, the material may expand by at least about 5%, 10%, 20%, 25%, 30%, 50%, 75%, 100%, 150%, 200%, 250%, or 300% of its original size. Likewise, the material by compress by at least about 10% to at least about 99% of its original size. For example, the material may compress by at least about 5%, 10%, 20%, 25%, 30%, 50%, 75%, or 99% of its original size.

The graft and/or material may be bioresorbable, or non-resorbable. While non-resorbable grafts may necessitate the need for an additional operative procedure, clinician control over the duration of time the graft remains intact could allow for increased chondral or osteochondral integration. The graft could be constructed to remain implanted for an indefinite period of time without negatively interfering in any biological processes or causing the patient pain.

The graft and/or porous material may be composed of synthetic or natural material, or a combination of both. The natural material may be of human, animal, and/or plant origin. One naturally derived material useful in the grafts of the invention is made of demineralized bone matrix (DBM).

When the graft is made of DBM, the material may be processed to allow for variations in degree of mineralization throughout the graft. This may affect the compressible/expandable nature of the graft, so that its compressible nature may vary with location in the graft. This may be particularly advantageous in reconstructive procedures where structural rigidity of a graft is imperative.

A devitalized cartilage matrix may be produced using a process similar to that used to create Osteosponge™. The starting material could be either cartilage only or could be an osteochondral core. Any source of cartilage cells could be used. Either could be processed to achieve a material that is expandable and/or compressible and appropriate for cartilage repair.

Besides the porous material for cartilage growth, the graft may include other portions, for example, a bone portion. The graft may consist of a cartilage portion extending or not into the bone portion of the defect. The graft may also consist of a bone portion extending into the cartilage portion of the defect. Alternatively, the graft may consist of two separate implants used in the same defect; a cartilage-appropriate portion and a separate bone-appropriate portion. The two portions may be separated by a membrane to prevent fluid migration or may be used as delivery of biological factors.

The graft may be seeded with one or more types of cells prior to, at the time of, or after implantation. “Seeding” the graft with cells refers to the process of inserting, or placing, one or more types of cells into, or onto, at least a portion of the graft. The cells can be placed in or on the porous material of the graft.

Suitable cells for seeding the graft include any kind of cartilage producing cells, or any kind of cells which may have a therapeutic affect, either in the graft or by migration out of the graft. Suitable cells include, but are not limited to embryonic stem cells, stem cells, bone marrow cells, mesenchymal cells, progenitor cells, synovium cells, synovial fluid cells, chondroblasts, chondrocytes, osteoblasts, or combinations of these cells.

Any cells added to the graft can be retrieved from various sources, including the patient to be treated, other patients of the same species, pools of cells from other patients or animals, individual animals and commercially available cell lines. Cells may be unaltered and seeded onto grafts immediately after removal from the source or remain in culture until being added to the graft. The cells may be allogenic, autogenic, or xenogenic to the patient to be treated. Combinations of cells may be used.

The graft can be used as an ex vivo matrix for cell growth and/or may be implanted in situ into a cartilage defect as an in vivo matrix for cell growth. The invention also comprises a graft produced by culturing with cells.

The graft can be cultured with appropriate cells ex vivo until cartilage forms and then implanted, cultured with appropriate cells ex vivo and implanted before full cartilage formation, or implanted without any culturing step at all.

One or more biological agents may be added to the graft. By “biological agent” it is meant any agent that has, or produces, biological, physiological and/or pharmaceutical activity upon administration to a living organism. These biological agents may be added to the graft at any time, for example, before, during or after implantation.

The graft can have varying degrees of biological agent content. The presence of biological agents can be controlled such that growth factor content is maximal or negligible. Biological agent content may vary with depth or location.

Suitable biological agents include, but are not limited to, growth factors, cytokines, antibiotics, strontium salts, fluoride salts, calcium salts, sodium salts, bone morphogenetic factors, chemotherapeutic agents, angiogenic factors, osteoconductive agents, chondroconductive agents, inductive agents, painkillers, proteins, peptides, or combinations thereof.

Growth factors that can be added to the graft include platelet derived growth factor (PDGF), transforming growth factor beta (TGFβ), insulin-related growth factor-I (IGF-I), insulin-related growth factor II(IGF-II), beta-2-microglobulin, bone morphogenetic protein (BMP), fibroblast growth factor (FGF), interleukin-1β (IL-1β), hepatocyte growth factor (HGF), cartilage derived morphogenetic protein (CD-MP), growth differentiation factors (GDFs), platelet-rich-plasma (PRP), or combinations of growth factors.

Chondroinductive agents include prostaglandin E2, thyroid hormone, dihydroxy vitamin D, ascorbic acid, dexamethasone, staurosporine, dibutyrl cAMP, concavalin A, vanadate, FK506, or combinations of different chondroinductive agents. Antibiotics include tetracycline hydrochloride, vancomycin, cephalosporins, and aminoglycocides such as tobramycin, gentamicin, and combinations thereof. Pain killers include lidocaine hydrochloride, bipivacaine hydrochloride, ketorolac tromethamine and other non-steroidal anti-inflammatory drugs.

The biological agent added to the graft can also be a protein or combinations of proteins. For example, proteins of demineralized bone, bone protein (BP), bone morphogenetic protein (BMP), BMP5, osteonectin, osteocalcin, osteogenin, or combinations of these proteins can be added to the graft.

Other suitable biological agents include cis-platinum, ifosfamide, methotrexate, doxorubicin hydrochloride, or combinations thereof.

The graft can be implanted dry or hydrated with liquids before, during or after implantation. Examples of liquids include, but are not limited to water, saline, and bodily fluids (blood). All or only part of the graft (for example, the porous material or part thereof) may be hydrated. The hydration by done by any method, including dipping, sprinkling, full or partial submersion, or running under a faucet. The graft may be exposed to the liquid for an instant up to several hours or several weeks, and can be stored in a liquid indefinitely until implantation.

The method of the invention can be used to treat any cartilage defect, whether it is in elastic cartilage, fibrocartilage, or hyaline cartilage. For example, the method could be used for cartilage repair in joints, such as a knee, ankle, hip, shoulder, elbow, temporomandibular, sternoclavicular, zygapophyseal, and wrist; or any other place where cartilage is found, such as the ear, nose, ribs, spinal column, pelvis, epiglottis, larynx, and windpipe. The graft may also be used in rhinoplasty procedures, including but not limited to reconstruction via a dorsal septal graft. The graft may be used to repair cartilage during a microtia-atresia surgical correction or in other types of auricular reconstructive procedures, such as those secondary to trauma or cancer.

The graft of the invention can be used to repair cartilage in any patient in need thereof. By “patient” is meant any organism which has cartilage, including, but not limited to humans, monkeys, horses, goats, dogs, cats, and rodents.

One graft may be used alone to fill the defect, or multiple grafts may be combined to fill one defect (similar to the mosaicplasty technique). In addition, the graft may be used to compliment other cartilage repair procedures, including autograft, allograft, or mosaicplasty procedures. The graft of the invention may be implanted at the same time, before, or after other cartilage repair procedures.

The expandable/compressible material may be used to fill small gaps left during the other procedures. The graft can be used to fill either the donor or the recipient sites in mosaicplasty-like procedures, and can be used either alone or in combination with other materials, including allografts, autografts, other biomaterials or other grafts.

The graft can be produced in various shapes and sizes. The graft may be produced in a geometric shape, such as a flat sheet, square, rectangle, cylinder, pentagon, hexagon, T-shape, cone, or circle. The graft may also be produced to match the shape of all or part of an anatomical feature, such as an ear, nose, joint, knee, ankle, hip, shoulder, elbow, temporomandibular, sternoclavicular, zygapophyseal, wrist, rib, spinal column, pelvis, epiglottis, larynx, or windpipe.

A surgeon may alter the size of the graft material prior to implantation by means of scissors or some other instrument or device used for cutting. This gives the clinician the operative flexibility to customize the fit of the invention without detriment to the patient or the graft itself.

Prior to, after, or in the absence of compression, the graft can be shaped by the clinician to match any anatomical intricacies of the surgical implantation site. The graft can then be implanted, either dry or hydrated, via a procedure such as “press fit.” The graft can be compressed prior to implantation, or can be implanted without compression. The graft material may expand to substantially fill the defect after implantation.

An undersized void can be created in the cartilage and possibly the adjacent bone where a defect is identified. For articulating joints, the surgeon creates a defined defect in the articulating joint where fibrillation or a cartilage defect was identified. The defect may be chondral or osteochondral. The graft, which can be oversized compared to the defect, may be compressed and implanted into the defect, either dry or hydrated. The graft may be compressed by any method, including by hand, by squeezing though a conical tube of a desired size, or via surgical instrument.

The graft may fill any void space by expanding to substantially fill the total volume of the defect. The constraint created by the undersized defect creates an increased press-fit with the surrounding tissue, enhanced integration and the elimination of micromotion. The graft may also be implanted without a press-fit or interference fit but will expand after implantation due to hydration with body fluids.

The graft may be merely press fit into the defect area or an anchor can be used to affix the graft to the defect. Anchors include plates, nails, screws, pins, adhesives, organic glues (such as fibrin glue), clotting materials or any other material known to be suitable for affixing cartilage or bone grafts. More than one type of anchor may be used to affix the graft to the cartilage defect site.

Because the graft can be compressible in all dimensions, it can be compressed to fit into small articulating joints, such as the hip. Thus, the ability to be compressed in three dimensions allows a graft to be used in the repair of cartilage defects of the hip or other articulating joints or during arthroscopic surgeries.

Another embodiment of the invention is a variation of the press-fit technique. One challenge of certain procedures, particularly in the area of oral surgery is primary closure of the wound site post-osseous graft. This occurs when an osseous defect receives a graft intended to serve as a matrix for osseous regeneration. The surgeon faces the challenge of suturing the epithelial layer over the graft. The graft can be compressed and encapsulated in a bio-resorbable or non-resorbable capsule. The capsule can be made in a varying array of shapes and sizes. The capsule can be slightly smaller than the defect being grafted, or can be compressed to a size slightly smaller than the defect to be grafted.

The capsule can be implanted into the defect and the surgeon sutures the epithelial tissue over the capsule inside of the defect creating a snug fit. The fit of the capsule should be tight enough to remain in place for suturing, but not occupy so much space as to make primary closure a challenge.

After closure, the blood and fluids in the grafted defect can initiate bioresorbtion of the capsule allowing the material to expand to its full size within the defect. The fit of the material becomes tight with the borders of the defect, minimizing any micromotion within the defect.

The surgeon selects the size of the capsule and hydrated material based on the anatomical defect. Multiple capsules could be used if necessitated by the anatomical defect.

Instrumentation or imaging techniques to measure and match the cartilage defect and/or surgical instruments used in conjunction with graft implantation may be packaged with the graft as a kit.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention.

The entire disclosure of all references discussed herein is hereby incorporated by reference herein.

### EXAMPLE 1

Osteosponge™ (Bacterin) was used as the graft material in all examples.

In this example, an in vitro study was performed to quantify the expansion of a demineralized bone matrix sponge when hydrated with commercially available 1× phosphate buffered saline (PBS).

Hydration was conducted by manually compressing and submerging the sponge in PBS, until pliable. In an effort to reproduce surgical conditions, the sponge was hydrated at room temperature.

The diameter, thickness and volume of the sponge were measured 3 times. Measurements were taken when the sponge was dry, immediately after being hydrated for 1 hour, and immediately after being hydrated for 2 hours.

The percent change in the diameter, thickness and volume were calculated by comparing both the 1 hour measurements and 2 hour measurements to the dry measurements. The sponge expanded ˜15% in diameter, ˜11% in thickness and ˜45% in volume. The measurements taken at 1 hour and 2 hours were statistically equivalent. See Table 1.

TABLE 1

| | Diameter (mm) | Thickness (mm) | Volume (mm³) |
|---|---|---|---|
| dry | 5.01 ± 0.12 | 8.18 ± 0.30 | 161.14 ± 6.27 |
| hydrated | 5.77 ± 0.13 | 9.11 ± 0.59 | 238.48 ± 24.46 |
| 1 hour | | | |
| hydrated | 5.74 ± 0.13 | 9.04 ± 0.33 | 233.95 ± 16.23 |
| 2 hours | | | |
| % change | 14.57% ± 3.71% | 10.59% ± 3.68% | 45.37% ± 12.07% |

### EXAMPLE 2

In vitro studies were also conducted to demonstrate the ability of demineralized bone matrix (DBM) sponges to support chondrogenesis. The sponges were divided into two groups: sponges containing cells and sponges without cells.

Chondrocytes were harvested from the rear joints of goats under the age of 3 months old. The articular cartilage was harvested within 24 hours of death. Articular cartilage was collected from the patellar groove, femoral condyle, and patella.

Throughout harvesting, the tissue was bathed in PBS containing gentamicin (25 ug/mL). Cartilage tissue was digested using 0.2% collagenase (Worthington collagenase type 22, 2 mg collagenase per mL culture medium) for approximately 18 hours at 37° C. while shaking in an orbital shaker. The resulting cells were pelleted by centrifugation at 200g for 10-15 minutes and strained through a 70 um cell strainer to separate the cells from cell debris and tissue fragments.

Following harvesting, the chondrocytes were seeded onto the sponges in 1 mL of cell culture medium (DMEM with 25 ug/mL gentamycin and 10% fetal bovine serum), at 37° C. in 24-well plates. The sponges were placed into the plates and the cell solution was placed on top of the sponges at a cell density of 30 million cells/cm³. The plates were shaken at 200 rpm for 18 hours.

After 18-24 hours of seeding, five cell-laden sponges from each experimental group were stained with MTT to assess cell distribution. Other cell-laden sponges were cultured in 6 well plates with 10 mL of culture medium comprising 10% FBS, 25 ug/ml of gentamicin and 50 ug/mL ascorbic acid for up to 6 weeks. The constructs were refed two to three times a week, and full refeeds were used (where all of the media is removed).

Sponges were analyzed at 3 weeks and 6 weeks for biochemical content, matrix uniformity and biomechanical properties.

DNA and glycosaminoglycan (GAG) content were assessed based on Hoechst 33258 and DMMB assays. GAG is a major constituent of the extracellular matrix of articular cartilage and indicates cartilage formation. See Table 2.

TABLE 2

| | % GAG (wet weight) |
|---|---|
| week 1 (N = 7) | 1.55 ± 0.53 |
| week 3 (N = 7) | 4.54 ± 2.04 |
| week 6 (N = 4) | 3.19 ± 0.50 |

The results prove a significant increase in GAG content in the cell-laden grafts thus indicating the presence of cartilage formation in the grafts containing chondrocytes.

Cross sections of grafts, both with and without chondrocytes, were stained with Safranin-O after six weeks in culture. Safranin-O is a red dye stain used to stain cellular nuclei in histological applications. Histological analysis of the samples revealed a cartilage like uniformity in the sponges containing chondrocytes, further supporting chondrogenesis in the cell-laden grafts.

### EXAMPLE 3

In the first in vivo study, the grafts were successfully implanted into defects created in the lateral and femoral condyle and trochlear grooves of goats. The femoral condyle was chosen because of its heavy weight bearing characteristics while the lateral groove was chosen because it is a lesser weight bearing site.

Tubular chisels were used to create and remove chondral and osteochondral cores measuring 4.5 mm in diameter. The remaining defects served as the implantation sites for grafts.

One graft consisting of DBM was hydrated with saline and implanted into each defect. Some grafts were combined with approximately 100-300 ul of fibrin glue according to manufacturer's instructions. Success was determined based on the ease of implantation, and whether the implanted grafts remained in the defect for the duration of the study.

### EXAMPLE 4

A second in vivo study examined the fixation of the grafts within an osteochondral defect after implantation.

The graft was initially hydrated with PBS. The graft was then compressed from a hydrated diameter of ˜6 mm in diameter into focal osteochondral defects of ˜4.5 mm. The grafts and defects were both ˜8 mm in depth.

Using the press-fit technique, the grafts were implanted into the lateral trochlear grooves and the medial femoral condyles of goats.

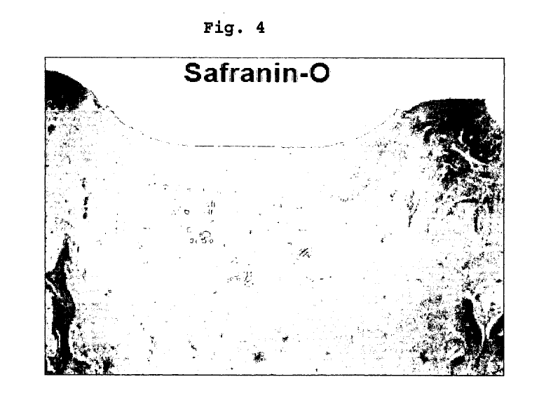

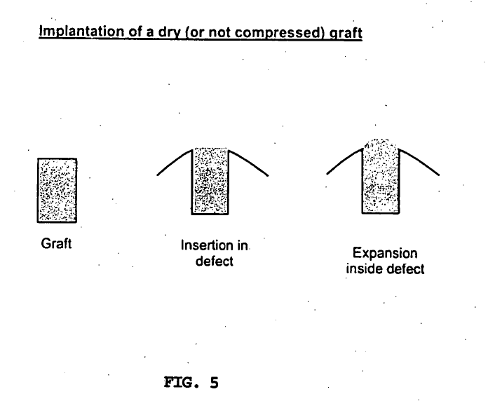

After 3 weeks, the animals were sacrificed, and the joints were histologically analyzed for the presence of the sponge. Safranin-O staining of cross sections of the joints containing sponges revealed remnants of the sponge still present in the sites of implantation. See FIG. 4.

### EXAMPLE 5

A third in vivo study examined the repair of focal osteochondral defects post-implantation. Results were examined after three months of implantation.

Two groups were studied in the current example and each group contained eight replicates. Each replicate was a goat femur containing two defects in the medial femoral condyle and two defects in the lateral trochlear groove.

For Group 1, osteochondral defects that received DBM grafts were compared to analogous defects that received autografts. For Group 2, osteochondral defects that received DBM grafts were compared to analogous defects that received microfracture.

Four defects were created using tubular chisels to create and remove osteochondral cores 4.5 mm wide and 8 mm deep. Two defects were made in the medial femoral condyle and two defects were made in the lateral trochlear groove of each replicate. The osteochondral grafts harvested from the first site at the condyle and the first site at the trochlear groove were disposed of. For Group 1, the grafts harvested from the second sites at the condyle and trochlear groove were implanted into the first defects at their respective locations. For Group 2, the full-thickness defect (articular and calcified cartilage removed) was created with a diameter of 4.5 mm. The defect was created using a tubular chisel, #15 scalpel blade and a currette. An awl was to create small holes in the subchondral bone, simulating microfracture in the goat. Perforations were made uniformly within the defect sites at an approximate depth of 3 mm.

The grafts, having initial hydrated diameters of ˜6.5 mm and widths of ˜8.5 mm, were compressed and implanted into the focal osteochondral defects employing the press-fit technique.

Post-implantation, the sponges protruded 0.5 mm proud to the adjacent cartilage. This technique is thought to aid in chondrogenesis.

After 3 months, the animals were sacrificed, and the joints were histologically analyzed for the presence of the sponge. Safranin-O staining of cross sections of the sites containing sponges revealed remnants of the sponge present in the implantation sites.

The presence of remnants of the sponges 3 months post-surgery proves the effectiveness of the technique in creating a sufficient fit between a sponge and the associated osseous or osteochondral defect. In comparison to the autograft-filled defects, the repair tissue in the DBM-filled defects shows a histological integration with the adjacent cartilage (FIGS. 3A and 3B), while the autografted sites demonstrated a gap between the osteochondral defect and the adjacent cartilage (FIG. 3C).

TABLE 3

| Group # | Objective | Timepoint | Defect #1 | Defect #2 |
|---|---|---|---|---|
| 1 | Effect of | 3 months | Scaffold in | Autograft |
| | scaffold in | | osteochondral | (positive |
| | osteochondral | | defect | control) |
| | defects | | | |
| 2 | Effect of | 3 months | Scaffold in | Microfracture |
| | scaffold in | | osteochondral | (clinical |
| | osteochondral | | defect | control) |
| | defects | | | |

1. A method for repair of a cartilage defect in a patient in need thereof comprising implanting a graft into said cartilage defect, wherein said graft comprises a porous material, wherein said material is expandable or compressible.

2. The method of claim 1, wherein said material comprises a material selected from the group consisting of demineralized bone matrix, allogenic tissue, xenogenic tissue, and synthetic material.

3. The method of claim 1, wherein said material is expandable.

4. The method of claim 3, wherein said material is expandable by at least 10%.

5. The method of claim 1, wherein said material is compressible.

6. The method of claim 5, wherein said material is compressible by at least 10%.

7. The method of claim 1, wherein said graft further comprises at least one biological agent.

8. The method of claim 1, wherein said implantation is performed before, after, or at the same time as a procedure selected from the group consisting of mosaicplasty, autograft, or allograft.

9. The method of claim 1, wherein said material is both expandable and compressible.

10. The method of claim 1, wherein said graft further comprises cells.

11. The method of claim 10, wherein said cells are selected from the group consisting of mesenchymal stem cells, chondrocytes, osteoblasts, and chondroblasts.

12. The method of claim 10, wherein said cells are added to said material prior to implantation, at the time of implantation, or after implantation.

13. The method of claim 12, wherein said cells are cultured ex vivo with said material prior to implantation.

14. The method of claim 1, where said defect is located in a site selected from the group consisting of knee, ankle, hip, shoulder, elbow, temporomandibular, sternoclavicular, zygapophyseal, wrist, ear, nose, ribs, spinal column, pelvis, epiglottis, larynx, and windpipe.

15. The method of claim 1, where said material is compressed to a smaller size than the defect to be repaired prior to implantation.

16. The method of claim 1, wherein said material expands to substantially fill the defect on implantation.

17. The method of claim 1, wherein said material is press fit into the defect.

18. The method of claim 1, wherein said material is fixed to the defect with an anchor.

19. The method of claim 18, wherein said anchor is selected from the group consisting of glue, adhesive, a screw, and a nail.

20. The method of claim 1, wherein said graft is hydrated prior to implantation, at the time of implantation, or after implantation.
